(19) United States
(12) Patent Application Publication
(10) Pub. No.: US 2010/0286569 A1
(43) Pub. Date: Nov. 11, 2010

(54) MASSAGE APPARATUS

(75) Inventor: Masaki Nagano, Osaka (JP)

Correspondence Address: BACON & THOMAS, PLLC, 625 SLATERS LANE, FOURTH FLOOR, ALEXANDRIA, VA, 22314-1176, US

(73) Assignee: Panasonic Electric Works Co., Ltd., Osaka (JP)

(21) Appl. No.: 12/810,890

(22) PCT Filed: Dec. 23, 2008
(86) PCT No.: PCT/IB08/03623

§ 371 Date: Jun. 28, 2010

(30) Foreign Application Priority Data
Dec. 28, 2007 (JP) 2007-341335

(51) Int. Cl. A61B 5/103; A61H 7/00; A61H 15/00

(52) U.S. Cl. 600/587; 601/84; 601/112

(57) ABSTRACT

A massage apparatus includes: a supporting body 1 that supports a body of a massage recipient on his front side and a massage mechanism 2 that causes a massage head 28 which is disposed behind the supporting body 1 to massage a back of the body of the massage recipient. The massage apparatus further includes a contour measurement unit 3 that measures contour of the body of the massage recipient supported by the supporting body 1, and the massage mechanism 2 includes a controller C that controls the movement of the massage head 28 based on information of the body contour obtained by the contour measurement unit 3. The massage apparatus can give a suitable massage to the back of the massage recipient in accordance with a different size of the back different per each massage recipient and a position of a part to be massaged depending on the size.

### TECHNICAL FIELD

The present invention relates to a massage apparatus, and more particularly relates to a massage apparatus that gives a massage to a body of a human to be treated (hereinafter called “a massage recipient”).

### BACKGROUND ART

Conventionally, various types of massage apparatuses have been provided in the form of a massage chair having a seat and a back rest. Such a massage chair accommodates a massage unit within the back rest 80 for massaging a back of a body (hereinafter a back refers to a back of a body unless specified otherwise) of a massage recipient.

As shown in FIG. 17, a back rest 80 of a massage chair includes a frame 87 with an opening at a center portion thereof, a massage head 81 disposed within the opening in the frame 87, and a back rest cover 89 that is to be in direct contact with a back of a massage recipient. When the massage recipient leans on the back rest 80, the frame 87 supports the back of the massage recipient. In order to enlarge a massage area, it is necessary to increase the size of the opening in the frame 87, which is a movable range of the massage head 81, because the massage head 81 moves within the opening in the frame 87. When the size of the opening is increased, however, the back rest 80 may fail to support the body of the massage recipient sufficiently. There is a tradeoff relationship between the size of the opening and the supporting of the body. That is, if the size of the opening is increased for securing a large massage area, the back rest 80 will fail to support the body of the massage recipient sufficiently. On the other hand, if the size of the opening is decreased for supporting the body securely, the massage area will be decreased. Practically, it is difficult to enlarge the size of the opening beyond the entire back of the massage recipient. Therefore, it is very difficult to massage a peripheral part of the back, e.g., outer parts of the shoulders (outside of a shoulder blade), which have a great requirement as a massage parts.

Further, the massage head 81 presses the back of the massage recipient such that the recipient is pushed up from the back rest 80. In this case, an excessive force is sometimes applied on the back of the massage recipient because the back is pressed not only by the massage head 81 but also by the weight of the massage recipient. Still further, in the case where the massage head 81 retracts into the back rest 80, the recipient's body subserviently moves downward in response to the retraction. Therefore, the pressure applied on the back is not decreased immediately, even when the massage head 81 retracts. Furthermore, the pressure applied on the back varies in accordance with an inclination angle of the back rest 80 or the weight of the massage recipient. Therefore, even if a projecting amount of the massage head toward the back of the massage recipient is controlled precisely, the applied pressure will not vary in proportion to the projecting amount. As stated above, the conventional massage chair can not control the applied pressure precisely and appropriately.

Then, the applicant of the present invention proposed a massage apparatus of a forward-bent posture supporting type (hereinafter called “a forward-bent type”) as disclosed in Japanese Patent Unexamined Publication No. 2007-209404. This massage apparatus is composed of a supporting body 1 for supporting a massage recipient on his front side and the massage mechanism 2 massaging a back of the massage recipient, as shown in FIG. 18. The massage apparatus gives a massage by projecting a massage head 28 against the back while supporting the massage recipient on the front side. Accordingly, the entire back of the massage recipient can be released, thereby making it easy to massage outer parts of the shoulders. Furthermore, since the weight of the massage recipient is not applied on the massage head 28, no weight affects pressures applied by the massage head 28. Therefore, in the massage apparatus of a forward-bent type, it is possible to adjust the force applied on the back precisely by controlling a projecting amount of the massage head 28.

However, since there is an individual difference with respect to the size and the contours of the back, the location of the part to be massaged or the pressure to be applied thereto is different for each massage recipient. In order to cope with such differences in physical constitutions of the massage recipients, some massage apparatuses of chair types are equipped with a function of detecting a location (height) of a shoulder. The massage apparatuses, however, have no function of detecting a shoulder length or the like. Therefore, it has been difficult to detect outer parts of a shoulder appropriately, although the parts have a great requirement as a massage part. A massage apparatus of a forward-bent type with these functions has not been provided so far.

### SUMMARY OF THE INVENTION

In view of the above-stated problems, it is a major object of the present invention to provide a massage apparatus that supports a massage recipient keeping a forward-bent posture, the massage apparatus enabling a more effective massage to a back of the massage recipient.

That is, a massage apparatus according to a first aspect of the present invention includes:

a support capable of supporting a front face of a human body;

a massage mechanism that massages a back of the human body supported by the support, the massage mechanism including a massage head disposed with a space kept from the support and being movable toward the support to come into contact with the back of the human body; a driving source that moves the massage head, and a controller that drives the driving source to control a movement of the massage head; and

a contour measurement unit that measures a contour of the human body supported by the support.

The controller drives the driving source so as to control the movement of the massage head based on measurement values measured by the contour measurement unit.

The massage apparatus is capable of controlling the movement of the massage head and a massage area based on the contours of the human body obtained by the contour measurement unit, thus enabling a more effective massage suitable for parts to be massaged.

Further characteristics of the present invention and advantages brought thereby will be clearly understood from the best mode for carrying out the invention described below.

### BRIEF DESCRIPTION OF DRAWINGS

FIG. 1 is a perspective view of a massage apparatus according to a first embodiment of the present invention;

FIG. 2 is an exploded perspective view of a massage mechanism of the massage apparatus;

FIG. 3 is an explanatory view showing an operation of a contour measurement unit of the massage apparatus;

FIG. 4 is an explanatory view showing divided regions in a back of a body;

FIG. 5A is an explanatory view showing a maximum neck width and a width between lower points of shoulder blades, and FIG. 5B is a correlation diagram showing a correlation between these lengths;

FIG. 6A is an exploded perspective view of a rotation unit, and FIG. 6B is a cross-sectional view of the massage head and the rotation unit;

FIG. 7 is a flowchart showing an operation of the massage mechanism;

FIG. 8 is a time chart showing the operation of FIG. 7 by using measurement values of a force sensor;

FIG. 9 is a time chart showing the operation of FIG. 7 by using another measurement value of a force sensor;

FIG. 10 is an explanatory view showing an operation of changing moving patterns of the massage head;

FIG. 11 is a perspective view showing another example of the massage apparatus;

FIG. 12 is a perspective view of a massage apparatus according to a second embodiment of the present invention;

FIG. 13A is an explanatory view showing directions of muscle fibers and a moving direction of a massage head, and FIG. 13 B is a time chart showing measurement values measured by the force sensor when the massage head moves in an ideal direction;

FIG. 14A is an explanatory view showing directions of muscle fibers and a moving direction of a massage head, and FIG. 14 B is a time chart showing measurement values measured by the force sensor when the massage head moves out of the ideal direction;

FIG. 15 is a perspective view showing a typical massage head;

FIG. 16 is a perspective view showing a massage head having a shape like a human thumb;

FIG. 17 is a perspective view showing the conventional massage apparatus of a chair type; and,

FIG. 18 is a perspective view showing a conventional massage apparatus of a forward-bent supporting type.

### BEST MODE FOR CARRYING OUT THE INVENTION

Referring to the attached drawings, the following will describe preferred embodiments of a massage apparatus according to the present invention in details.

### First Embodiment

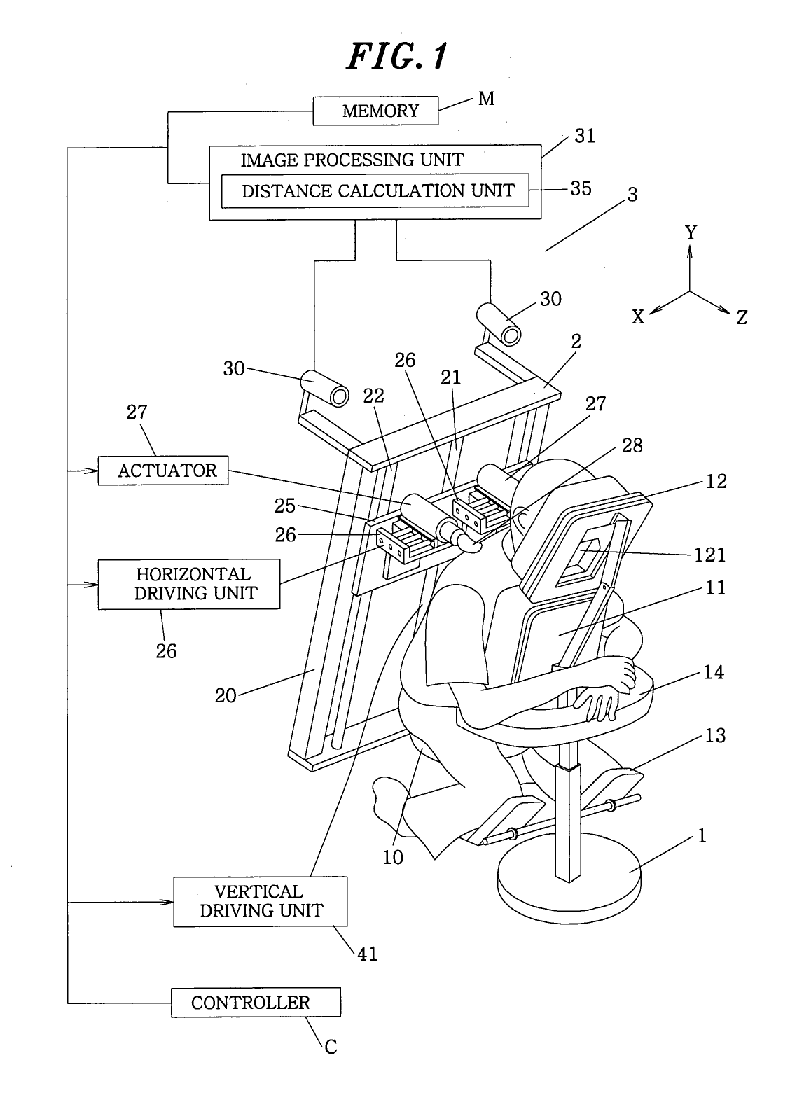

The following describes a first embodiment of the present invention, with reference to the attached drawings. As shown in FIG. 1, a massage apparatus in accordance with the present embodiment is provided with a supporting body 1 for supporting a massage recipient in a forward-bent seating posture and a massage mechanism 2 including massage heads 28, 28 for coming into contact with a back of the massage recipient and a controller C controlling a movement of the massage heads 28. The supporting body 1 includes a seat 10, a chest supporter 11 for supporting a front side of the massage recipient's chest, a head supporter 12 for supporting a front side of the massage recipient's head, a knee rest 13 for putting knees thereon, and an arm rest 14 for putting arms thereon. In order to ensure a breathing of the massage recipient supported at the front side of the head, the head supporter 12 has a through hole penetrating a whole thickness thereof at a position corresponding to a mouth and a nose of the massage recipient.

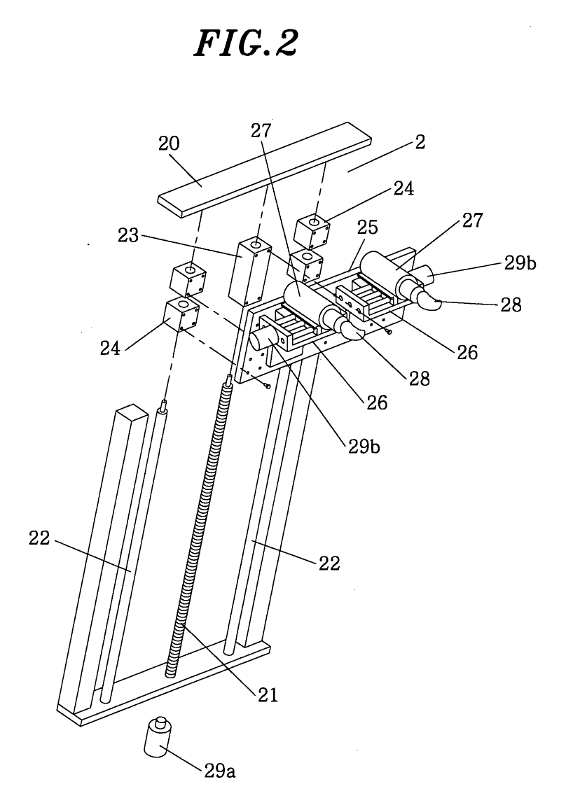

As shown in FIG. 1, the massage mechanism 2 disposed behind the supporting body 1 includes a vertical driving unit 41, a pair of horizontal driving units 26, 26, and a pair of actuators 27 to be operated as a projection and retraction driving unit. The vertical driving unit 41, as shown in FIG. 2, includes a frame 20 configured to be rectangular, a vertical driving shaft 21 disposed at the center of the frame 20 along the longitudinal direction, and a pair of right and left vertical guide shafts 22, 22 disposed so that the vertical driving shaft 21 is sandwiched therebetween in the frame 20. The surface of the vertical driving shaft 21 is threaded substantially over it's full length in the longitudinal direction, and is inserted into a feed nut 23 to be engaged with the threads. Each of the vertical guide shafts 22, 22 is inserted through two bearings 24, where each bearing 24 slides along the vertical guide shaft 22. The feed nut 23 and the four bearings 24 are attached to a surface (first surface) of a movable plate 25 by screws. The vertical driving unit 41 further includes a motor 29a attached to a lower end of the vertical driving shaft 21 and the motor 29a serves to rotate the vertical driving shaft 21. When the motor 29a rotates the vertical driving shaft 21, the movable plate 25 is moved vertically along the vertical driving shaft 21. Although the motor 29a is attached to the lower end of the vertical driving shaft 21, this may be attached to an upper end of the vertical driving shaft 21.

The horizontal driving units 26, 26 are attached to a second surface (an opposite side of the first surface on which the feed nut 23 and the bearings 24 are attached) of the movable plate 25. The actuator 27 is fixed to the horizontal driving units 26, 26 respectively, and a massage head 28 having a shape like a human thumb is attached to a tip end of a driving shaft of each actuator 27. Projecting the driving shaft projects the massage head 28 against the back of the massage recipient supported by the supporting body 1. The massage heads 28 are separated from the supporting body 1 by a space to be occupied by the massage recipient when the supporting body 1 supports the massage recipient.

The horizontal driving units 26, 26 move the actuators 27, 27 in the width direction of the frame 20, i.e., in the horizontal direction of the massage recipient. Each of the horizontal driving units 26, 26 includes a driving shaft having threads in its surface and a feed nut (not illustrated), as in the case of the configuration of vertical driving shaft 21 and feed nut 23. The rotation of the driving shaft of each horizontal driving unit 26 by a motor 29b moves the actuator 27 horizontally. As stated above, the horizontal driving units 26, 26 have the motor 29b respectively, enabling independent driving. Therefore, the massage heads 28, 28, which are attached to the actuators 27, 27, also can move horizontally, not depending on each other.

As shown in FIG. 1, the controller C controls each of driving sources, i.e., the actuators 27, the motor 29a of the vertical driving unit 41, and the motors 29b,29b of the horizontal driving units 26,26.

As described above, the massage mechanism 2 moves the massage heads 28, 28 in a three-dimension for massage by combining the vertical movement by the vertical driving unit 41, the horizontal movement by the horizontal driving units 26, 26, and the protrusion movement by the actuators 27, 27.

Since the frame 20 is fixed to be inclined in accordance with a forward-bent angle of the supporting body 1, the massage heads 28 can move perpendicularly to the back of the massage recipient who is supported by the supporting body 1 in a forward-bent posture. When the actuators 27 are driven in this condition, each of the massage heads 28 can apply a pressure perpendicularly to the back. Then, even when the applied pressure becomes larger, the body of the massage recipient will not slide out of the massage heads 28 because the chest supporter 11 supports the massage recipient's chest on the front side thereof sufficiently. Accordingly, the applied pressure by the massage heads 28 will not be decreased, so that the pressure can be surely applied to the back. Furthermore, the massage recipient is much more relaxed than when lying on his face, because the supporting body 1 can keep the massage recipient in a forward-bent posture. This is because, even though the chest supporter 11 is pressed by a reaction force of the applied pressure during the massage, the weight of the massage recipient is supported mainly by the seat 10. Therefore, the massage recipient feels no oppression or smothering caused by his own weight. Especially, when the massage recipient is relatively obese, such a relaxing effect will be enhanced. Since the massage apparatus of the present embodiment can give a massage in the relaxed condition, the massage effects by the massage heads 28 are significantly high.

Further, since no weight of the massage recipient is applied on the massage heads 28, the pressure applied to the back by the massage heads 28 relies on the projecting amount of the massage head 28 to the front side. Thus, it is easy to adjust the pressure applied to the back by controlling the projecting amount of the massage heads 28.

Moreover, since the supporting body 1 supports the body of the massage recipient, there is no need to provide the massage mechanism 2 with any more members required for supporting the body. Therefore, the horizontal driving units 26, 26 can drive the massage heads 28 up to a full width of the body in a horizontal direction. Thus, the massage area is significantly widened, thereby giving a massage to the entire back of the upper half body of the massage recipient.

This massage apparatus applies a pressure to the back by projecting the massage heads 28 to the supporting body 1. As described above, the massage heads 28 may be moved vertically and horizontally relative to the frame 20. In this case, the controller C moves the massage heads 28 not only in the direction perpendicular to the back of the massage recipient, i.e., in the pressing direction, but also in a vertical and horizontal direction along the back. By combining these movements, the controller C makes it possible that the massage heads 28 press the back while moving along the back. That is, the massage apparatus of the present embodiment can give the most effective massage, i.e., pressing the body while applying a force both in the surface being along the body surface and in the direction perpendicular to muscle fibers.

In order to realize the above-stated massage operation, the massage apparatus of the present embodiment includes a contour measurement unit 3 measuring contours of the massage recipient's body, and a memory M memorizing contour data of characteristic parts in an ordinary human body and information of a muscle fiber direction of a back. As shown in FIG. 1, the contour measurement unit 3 includes a pair of right and left imaging units 30, 30 fixed to the frame 20, and an image processing unit 31 processing images captured by the imaging units 30, 30. The imaging units 30, 30 are fixed to right and left top ends of the frame 20 respectively to generate a stereo image. As the imaging units 30, commercially available cameras may be used.

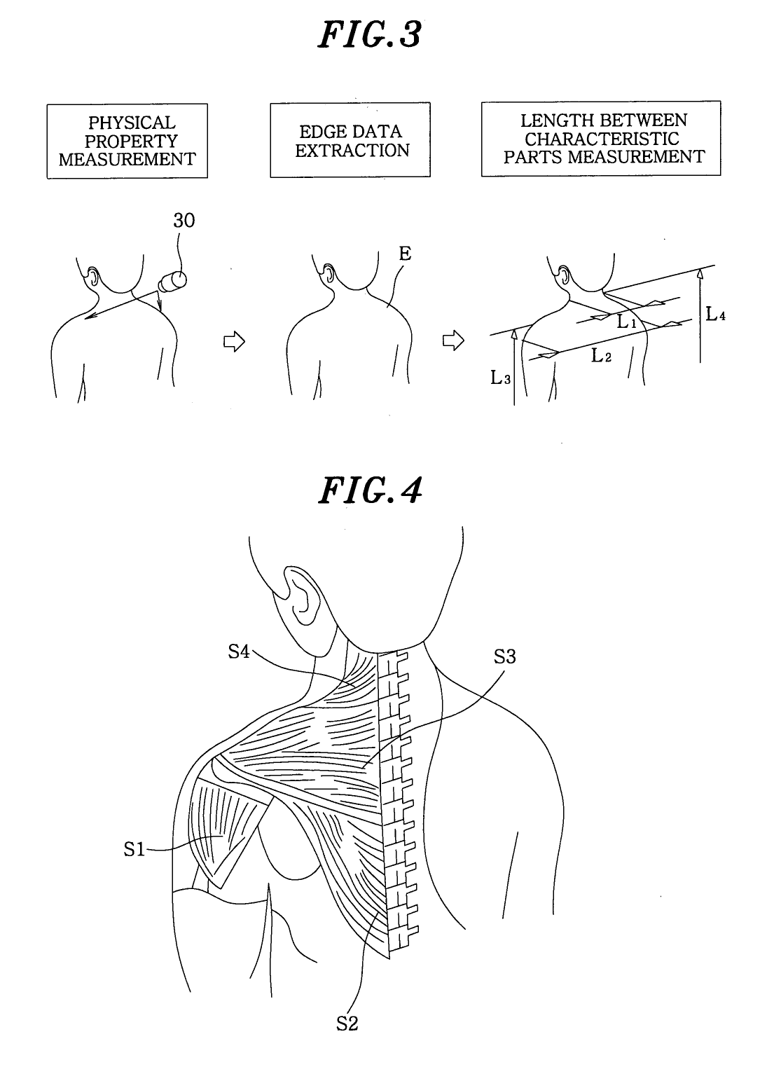

The imaging units 30, 30 capture images of the back of the massage recipient supported by the supporting body 1, and then the images are sent to the image processing unit 31. The image processing unit 31 extracts edge data E from the captured image as contour data shown in FIG. 3. Then, the image processing unit 31 compares the determined edge data E with the contour data stored in the memory M by pattern matching to specify a characteristic part (e.g., neck, shoulder). Subsequently, as shown in FIG. 3, the image processing unit 31 calculates a length of the thus specified part, i.e., a neck length L1, a shoulder length L2, a length L3 from the seat surface to the shoulder, a length L4 from the seat surface to the root of the neck, and so on based on the edge data E. Herein the image processing unit 31 further includes a length calculation unit 35 calculating a distance from the imaging units 30, 30 to the respective points of the back, where the distance can be calculated by utilizing a stereo image obtained by the two images which are captured by the imaging units 30, 30. Since positional relationships between the imaging units 30, 30 and the massage heads 28, 28 are determined by using driving states of the driving sources 27, 29a, 29b, the distance between the massage heads 28, 28 and the respective points of the back can be easily calculated.

The controller C acquires the edge data E obtained by the contour measurement unit 3, the information of the length between the characteristic parts of the body contour, and the information of the length from the massage heads 28 to the respective points of the back, and then causes the memory M to memorize the same. The controller C determines a massage area of the massage heads 28 based on the edge data E, and operates the driving sources 27, 29a, and 29b, such that the massage heads 28 can move around within the area.

Further, the controller C divides the back of the massage recipient into some regions as shown in FIG. 4. In the present embodiment, the back is divided into four regions. That is, a region S1 is a portion where triceps brachii muscles mainly exist, a region S2 is a portion where latissimus dorsi muscles mainly exist, a region S3 is a portion where trapezius muscles mainly exist, and a region S4 is a portion where levator scapulae muscles mainly exist. Such regions are preferably divided so that muscle fibers thereof have substantially uniform directions respectively as shown in FIG. 4. Note that the manner of diving into the regions is not limited to the example of FIG. 4, for example, the region S3 may be further divided into two regions.

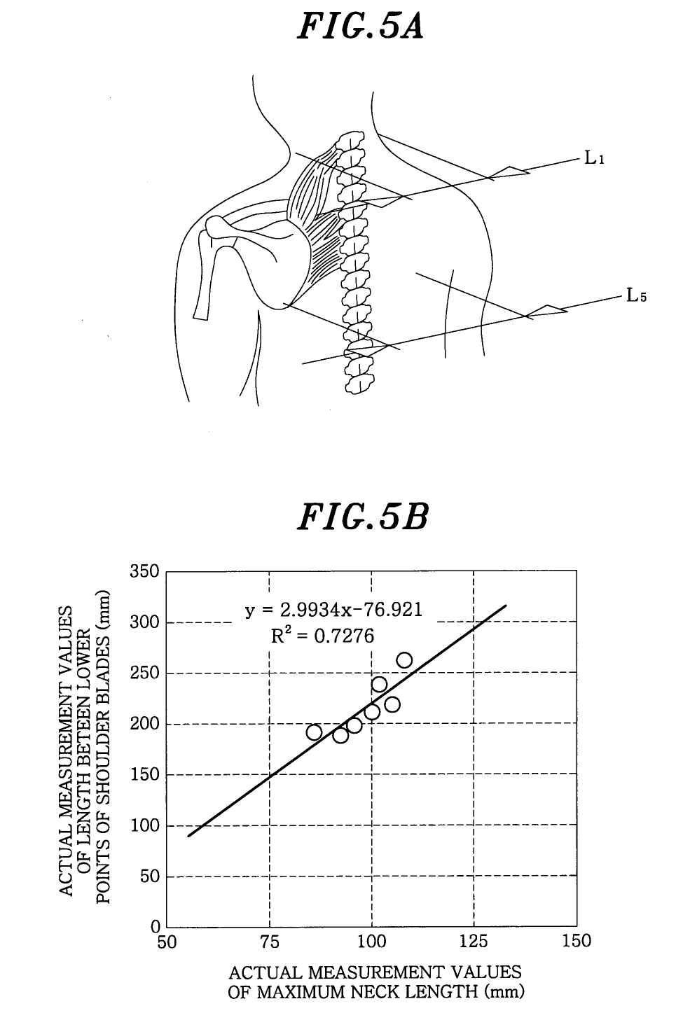

The regions where the respective kinds of muscle fibers locate in the back of the upper half body can be easily estimated from the edge data E and the length between the characteristic parts of the body contours. The controller C divides the back into such regions by utilizing the above-stated estimation. For instance, as shown in FIG. 5B, it has been verified that an actual measurement value of the maximum neck length L1 shown in FIG. 5A has a significantly high correlation with an actual measurement value of a length L5 between lower points of the shoulder blades. Thus, when the actual measurement value of the neck length L1, which is one of the characteristic parts of the body contours, is calculated by the image processing unit 31, then the value of the length L5 between lower points of the shoulder blades can be estimated from the correlation shown in FIG. 5B. In this way, the value of the length between the characteristic parts of the body contours is used to estimate a position where each kind of muscle fibers locates in the back of the upper half body.

An ideal massage is said to be an operation of pulling muscle fibers in the direction perpendicular to a muscle fiber direction (and in the direction along the body surface) while applying the force in the direction perpendicular to a surface of a body (e.g., in the direction pressing the body surface). This massage operation is considered to be preferable in terms of not only the massage effects but also the sensual aspects. Thus, when the back is divided into the regions S1 to S4 as stated above, the direction of the muscle fibers in each region S1 to S4 can be determined because the memory M memorizes the information of the muscle fiber direction of the each part. Further, the muscle fibers in the each region have substantially the same direction. By adjusting the moving direction of the massage heads 28 with respect to each of the regions S1 to S4, the above-stated ideal massage can be realized.

That is, the controller C knows the position where the massage heads 28 are located among the regions of S1 to S4, and then moves vertically and horizontally the massage heads 28 in a direction perpendicular to the muscle fiber direction while pressing the massage heads 28 to the back, thereby enabling the above-stated ideal massage.

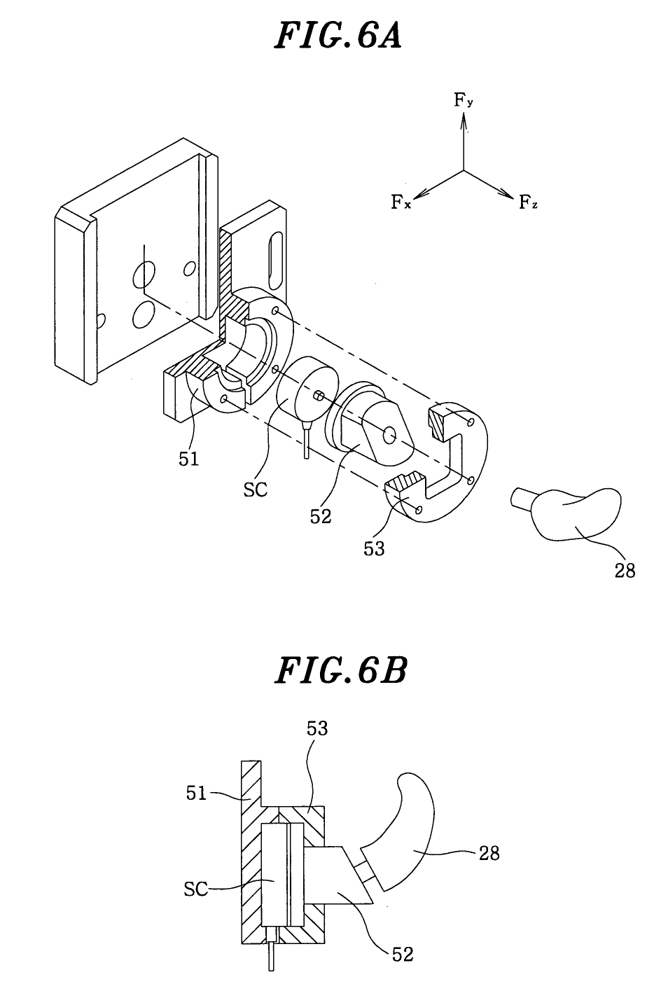

As shown in FIG. 6A, a force sensor SC is attached to each of the massage heads 28, where the force sensor measures a force applied to the massage heads 28. As shown in FIGS. 6A and 6B, the massage head 28 is attached to a float unit 52, and the float unit 52 and the force sensor SC are sandwiched between a base 51 and a cover 53 for being fixed. The force sensor SC measures the force applied to the massage head 28 through the float unit 52.

In the massage apparatus of the present embodiment, since the supporting body 1 supports the body of the massage recipient in a forward-bent posture, the weight of the massage recipient is not applied on the massage heads 28. Therefore, the force sensor SC can measure only a reaction force caused by the force applied to the back by the massage heads 28. When receiving the measurement values of the force sensor SC as feed-back input signals, the controller C regulates the pressure, which is applied to the back by the massage heads 28, to be suitable for the massage precisely and appropriately, without applying an excessive pressure to the back.

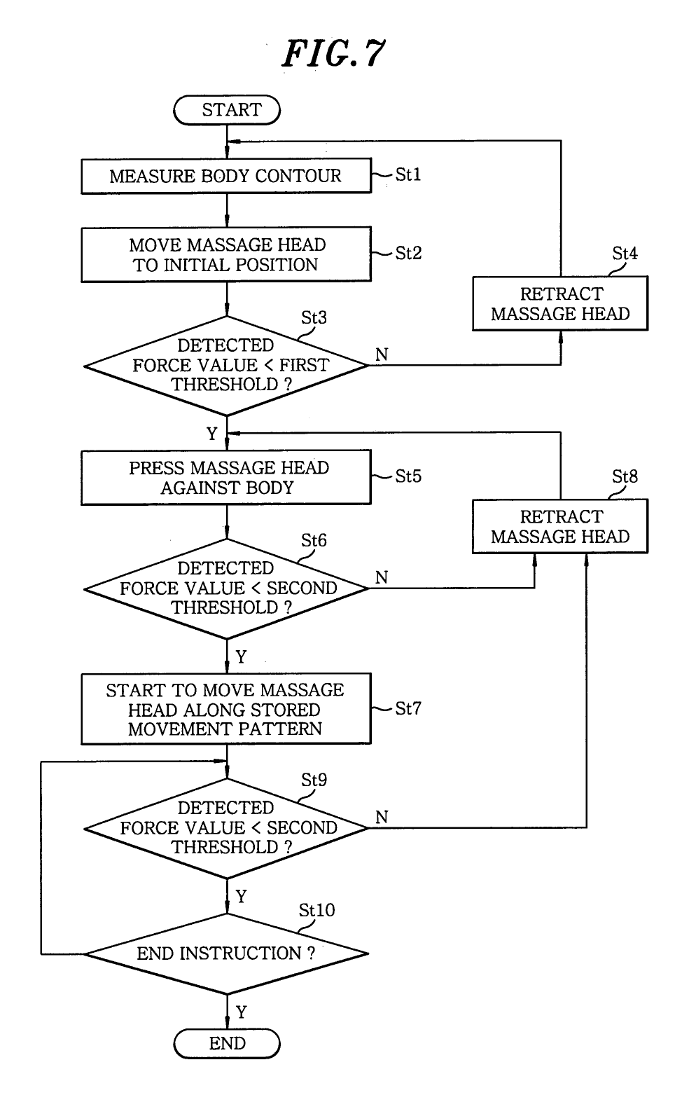

FIG. 7 is a flowchart showing the operation by the massage apparatus of the present embodiment. Herein, the massage heads 28 are located sufficiently away from the supporting body 1. Therefore, the massage recipient can easily enter the space between the supporting body 1 and the massage heads 28, then enabling the body to be supported appropriately by the respective parts of the supporting body 1. After the supporting body 1 has supported the massage recipient firmly, when the massage apparatus is started, the contour measurement unit 3 captures the back of the massage recipient by the imaging units 30, 30 to measure the body contour (Step St1). When the capture is finished, the controller C moves the massage heads 28 to an initial position (Step St2). The initial position is in the vicinity of the back but not in contact with the back. By comparing an output value of force sensor SC with a first threshold that is memorized in the memory M beforehand, the controller C judges whether the massage heads 28 is traveling correctly to the initial position or not (Step St3). More specifically, when the output value of the force sensor SC is smaller than the first threshold, it is shown that the massage heads 28 are traveling to the initial position without contacting any objects. On the other hand, when the output value of the force sensor SC is not less than the first threshold, the massage heads 28 are in contact with some object (in this case, the back). When being in contact with the back, then the massage heads 28 are moved backward, and the body contour is measured again (Step St1). Herein, there is a fear that the force sensor SC may detect the force caused by, for example, a swaying motion of the massage heads 28 when the massage heads 28 are traveling, even if being in contact with no objects. In order to prevent the controller C from misunderstanding that the above-stated detection is recognized to be in contact with the back, the first threshold is employed as a standard of the judgment. Steps St1 to St4 are operations for moving the massage heads 28 to the initial position after the massage apparatus starts.

Next, the operation of pressing the massage heads 28 to the back is started (Step St5). Herein, it is necessary to control a pressing amount of the massage heads 28 appropriately because the operation of the pressing directly affects the massage condition. Thus, in order to control the pressing amount, the controller C compares an output value of the force sensor SC with a second threshold which is stored in the memory M beforehand (Step St6). The pressing amount is determined to be proper when the output value of the force sensor SC is smaller than the second threshold. Further when the pressing amount is determined to be proper, the massage heads 28 are moved along the movement pattern that is memorized beforehand, thus giving a massage to the back (Step St7). When the pressing amount is too large and the output value of the force sensor SC exceeds the second threshold, an excessive pressure is applied to the back. Therefore, the massage heads 28 stop a further pressing and move backward so as to gradually decrease the pressure applied to the back (Step St8).

During the massage operation in the step St7 also, the output value of the force sensor SC is continuously compared with the second threshold, so as not to apply an excessive force to the back (Step St9). When the output value exceeds the second threshold, the massage heads 28 are moved backward to reduce the applied pressure (Step St8). On the other hand, when the output value is below the second threshold, the controller C judges whether an end instruction is input to the massage apparatus or not (Step St10). When the end instruction is not input, the procedure goes back to Step St9 to continue the massage operation. When the end instruction is input, the massage operation ends (Step Sal).

As stated above, according to the operation shown by the flowchart of FIG. 7, the massage heads 28 do not apply a pressure exceeding a predetermined threshold to the back. That is, thresholds can be determined appropriately based on physical properties of massage recipients and parts to be massaged, thereby preventing the massage heads 28 from applying an excessive pressure to the massage recipient.

The contour measurement unit 3 includes the two imaging units 30, 30. Since a stereo image is formed of the images captured by each of the imaging units, the length calculation unit 35 is able to calculate a distance from the contour measurement unit 3 to each part of the back, and further a distance from the massage head 28 to each part of the back. When the distance from the massage head 28 to each part of the back is known beforehand, the controller C can recognize the position in which the massage head 28 is in contact with the back before moving the massage head 28 to the back. Thereby, just until the massage heads 28 are in contact with the back, the controller C can move the massage heads 28 promptly without referring to the output signals of the force sensor SC, which means to reduce processing loads of a driving system including sensors. Further, even after the massage heads 28 are in contact with the back, the pressing condition on the back can be measured by using both a force strength detected by the force sensor SC and the distance from the back to the massage heads 28, which prevents an excessive pressing.

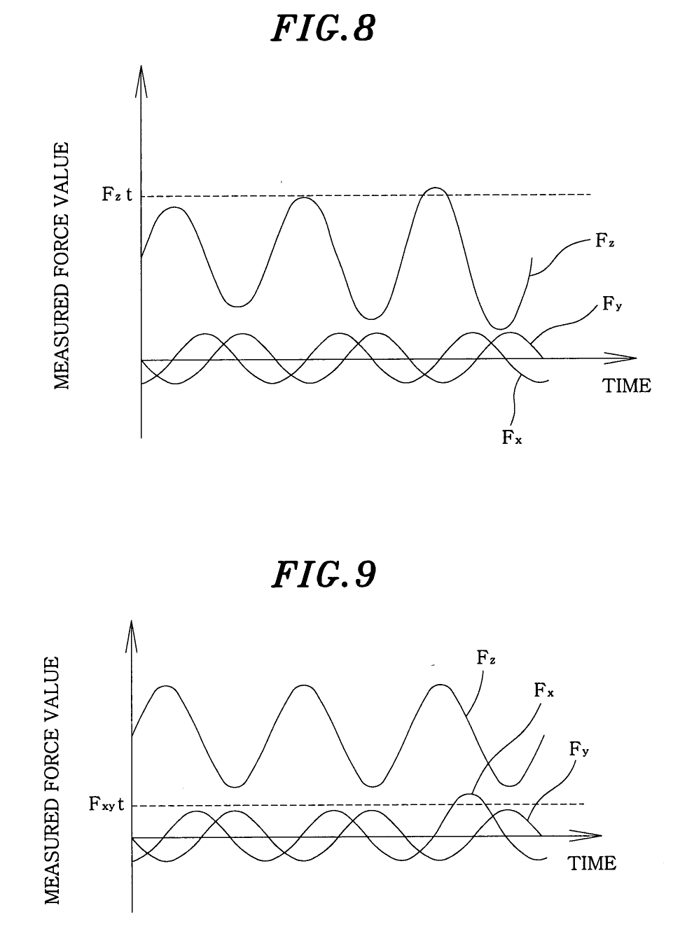

Herein, the force sensor SC detects forces in three-axis directions Fx, Fy, Fz, where movable directions X, Y, Z of the massage heads 28 are preferably aligned with the force directions Fx, Fy, Fz. That is, the pressing direction Z of the massage heads 28 is aligned with the Z direction of the force sensor SC, and the vertical and horizontal directions X, Y of the massage heads 28 are aligned with the X and Y directions of the force sensor SC. Herein, it is assumed that, for example, the massage is carried out such that the force Fz in the pressing direction of the massage head 28, the force Fx in the horizontal direction, and the force Fy in the vertical direction are varied as shown in FIG. 8. Here, a maximal value of the force Fz in the pressing direction increases gradually. When the force Fz exceeds a threshold Fzt indicated by the dashed line in FIG. 8, the force Fz in the pressing direction can be decreased immediately by moving the massage heads 28 backward in the Z direction. That is, the three-axial components of the forces detected by the force sensor SC agree with the three-axial ones of the moving directions of the massage heads 28. Therefore, if one force component exceeds the corresponding threshold, the massage heads 28 may be moved backward along the axial component of the moving direction corresponding to the force component. As a result, responsiveness can be improved because there is no need of complicated calculation for coordinate axis conversion.

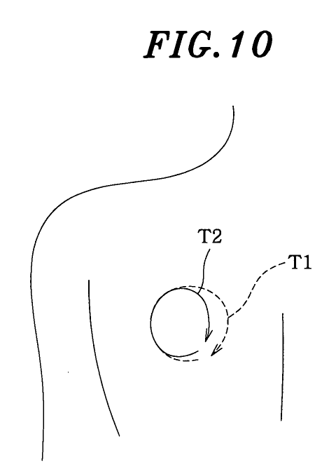

When the vertical and horizontal forces Fx, Fy exceed the threshold Fxyt indicated by the dashed line in FIG. 9, it means that the massage heads 28 are in contact with bones such as a shoulder blade. For instance, it is assumed that the massage heads 28 move along a circle pattern T1 as shown in FIG. 10. When the force Fx exceeds the threshold Fxyt as shown in FIG. 9, it means that the massage head 28 is in contact with a left end of the shoulder blade. In such a case, the controller C decreases a displaced amount of the massage head 28 in the horizontal direction (i.e., X-axis direction of the massage head 28), and changes the pattern T1 into pattern T2, thereby avoiding a massage for the human body part not to be massaged such as bones.

The memory M may store a position where the force exceeds the above-stated threshold Fxyt. With the reference to the position, the controller C can get know where an edge of a bone such as a shoulder blade locates. By utilizing this manner, the controller C can obtain the information more accurately where a position to be massaged (so-called a massage point that is related to the central and autonomic nervous systems) locates, and give a massage to the point. Furthermore, for example, the controller C enables the specified pressing operation which gets the massage heads 28 into a reverse side of a shoulder blade.

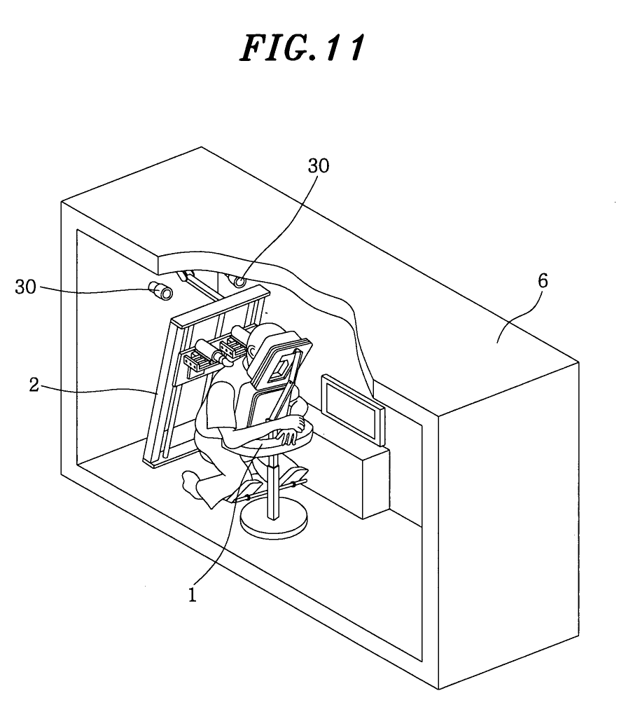

In the case where a camera is used as the imaging units 30, 30, and the massage recipient wears clothing, it may be difficult for the contour measurement unit 3 to extract the edge data E of the massage recipient because the data is affected by disturbance light and the dependence on the color and the brightness of the clothing. Therefore, as another example of the massage apparatus, it is preferable that the supporting body 1, the massage mechanism 2, the contour measurement unit 3 and an illuminance-adjustable lighting fixture (not illustrated) are installed in a box room 6 as shown in FIG. 11. The box room 6 is surrounded by walls made of light-blocking members, where the walls form a closed space. The contour measurement unit 3 is attached to a side wall of the box room 6, which corresponds to a backside of the massage mechanism 2. Thereby, the box room 6 can decrease the influences on the contour measurement unit 3 by the disturbance light from the outside, so that the edge data E can be extracted more accurately. Even when there is no difference in the color between the clothes and the wall surface, the brightness difference can be increased because the distance from the lighting fixture (not illustrated) to the clothing is different from the distance from the lighting fixture to each of the wall surfaces. As a result, it becomes easy to extract the edge data E.

As stated above, the massage apparatus of the present embodiment can give a massage to the entire back of the massage recipient. Further, the massage apparatus measures body contours, and can get information about a position of a body part to be massaged and a direction of muscle fibers per each massage recipient. Thereby, the massage apparatus can give more effective massages to the massage recipient.

### Second Embodiment

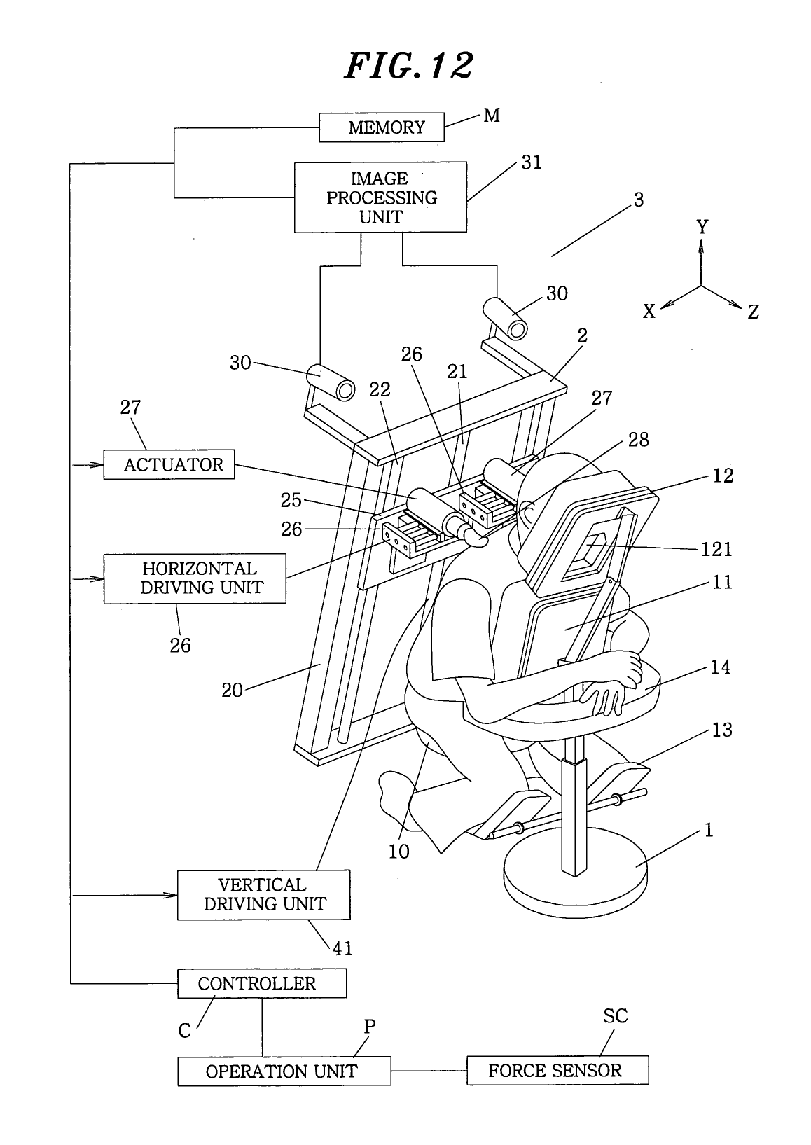

Referring to FIG. 12, the following describes a massage apparatus according to a second embodiment of the present invention. The same reference numerals are assigned to the same parts as those in the first embodiment, and their detailed description is omitted. The massage apparatus of the present embodiment includes massage heads 28 coming into contact with a body surface of a massage recipient, driving sources 27, 29a, and 29b moving the massage heads 28 in three dimensional directions, a force sensor SC detecting two-axial components of a force acting on the surface at least being along the body surface among forces acting on the massage heads 28, and an operation unit P determining a direction of muscle fibers of the back based on a ratio of the above-stated two-axial components of the force detected by the force sensor SC. That is, in this massage apparatus, when the massage heads 28 are moved in the two-axial directions (X, Y-axis directions) in the surface being along the body surface, the force sensor measures the force components in the above-stated X and Y axis directions, and the operation unit P determines the muscle fiber direction of the body surface by using the force components detected by the force sensor SC.

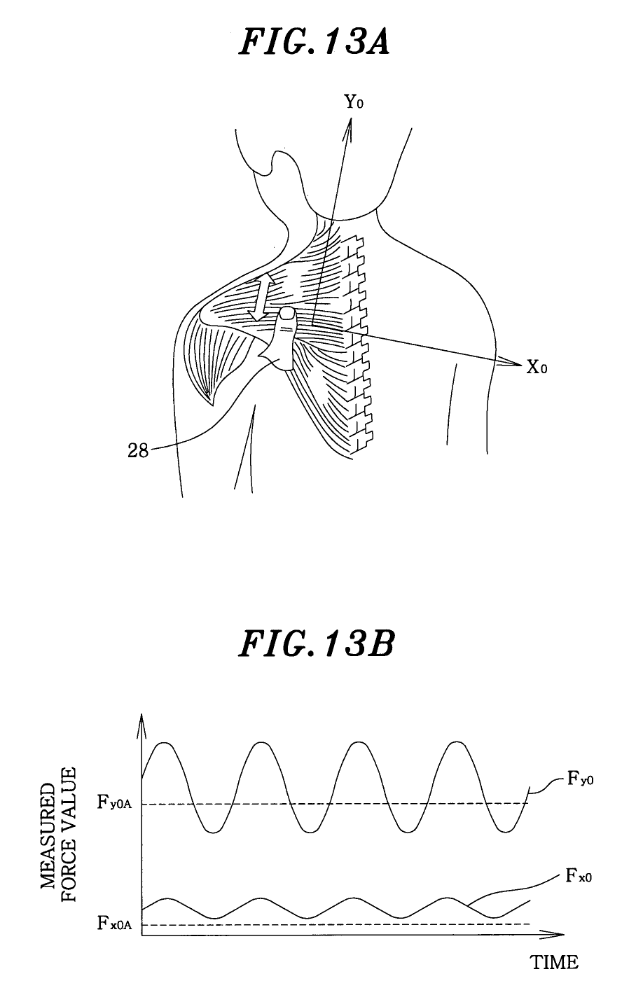

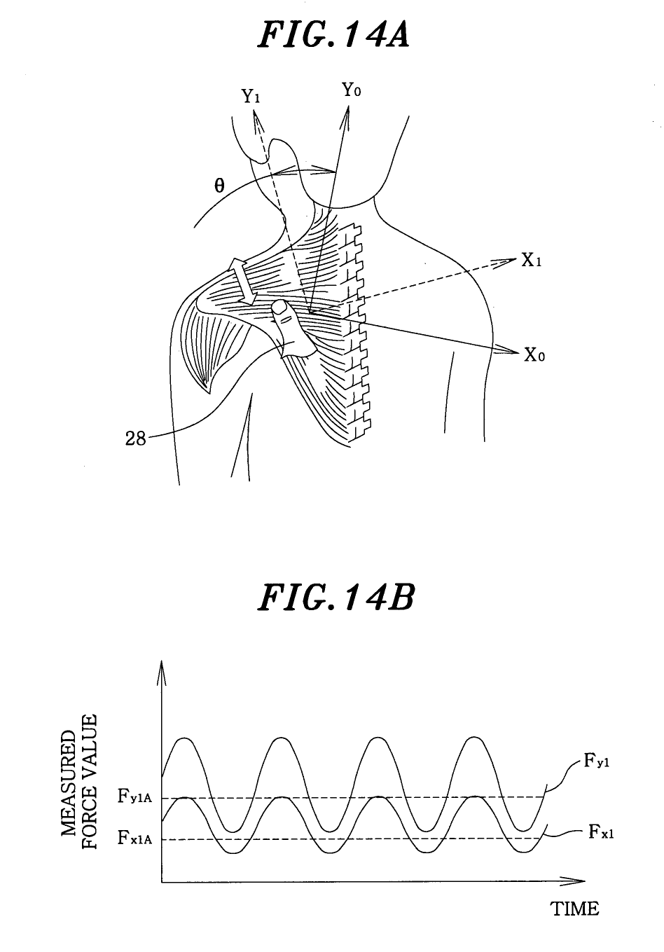

For instance, as shown in FIG. 13A, it is set that X₀ is the direction of muscle fibers of a part to be in contact with a massage head 28 and Y₀ is the direction perpendicular to X₀. When the massage head 28 is moved in Y₀ direction, a force component Fy₀ in Y₀ direction is, as shown in FIG. 13B, sufficiently larger than a force component Fx₀ in X₀ direction, both of which are detected by the force sensor SC. On the other hand, as shown in FIG. 14A, when the massage head 28 is moved in Y₁ direction that is deviated from Y₀ direction by an angle θ₀, a force component Fx₁ in X₁ direction value becomes closer to a force component Fy₁ in Y1 direction as shown in FIG. 14B, both of which are detected by the force sensor SC. Each of the following expressions shows a relationship between the measurement values by the force sensor SC and the deviation angle θ in each of the examples shown in FIGS. 13A and 14A. A deviation angle θ₀ in the example of FIG. 13A will be obtained by the following expression (1):

θ₀=tan⁻¹(Fx_{0A}/Fy_{0A}) (1)

where Fx_{0A} and Fy_{0A} are average values of Fx₀ and Fy₀ shown in FIG. 13B, respectively. Since Fy_{0A} is sufficiently larger than Fx_{0A}, the deviation angle θ₀ is closer to 0, thus satisfying θ₀<<ε where ε is a predetermined threshold.

A value of the deviation angle θ₁ in the example of FIG. 14A will be obtained by the following expression (2):

θ₁=tan⁻¹(Fx_{1A}/Fy_{1A}) (2)

where Fx_{1A} and Fy_{1A} are average values of Fx₁ and Fy₁ shown in FIG. 14B, respectively. Since Fy_{1A} is closer to Fx_{1A}, the deviation angle θ₁ is sufficiently large, thus satisfying θ₁>ε.

As stated above, the muscle fiber direction X₀ can be calculated from the ratio of the measurement values Fx and Fy detected by the force sensor SC. If the moving direction of the massage heads 28 is determined based on this calculation result, the massage to be preferred, which massage heads 28 are moved in the direction Y₀ perpendicular to the muscle fiber direction X₀, will be carried out more effectively.

In the case where the moving direction of the massage heads 28 deviates from the direction perpendicular to the muscle fiber direction by an angle θ, correction driving instruction values Vx′ and Vy′ to be output by the controller C to the drive sources 29a and 29b respectively, are given by the following expression (3):

$$[ \begin{matrix}{V}_{X}^{′} \\ {V}_{Y}^{′}\end{matrix} ] = [ \begin{matrix}cos θ & sin θ \\ - sin θ & cos θ\end{matrix} ] [ \begin{matrix}{V}_{X} \\ {V}_{Y}\end{matrix} ] \qquad ( 3 )$$

The controller C outputs these correction driving instruction values Vx′ and Vy′ to the driving sources 29a, 29b, respectively, whereby the massage heads 28 can move in the direction perpendicular to the muscle fiber direction, thus enabling effective massage. Herein, the driving instruction values Vx′ and Vy′ are stored beforehand in the memory M. When the controller C drives the driving sources 29a, 29b in accordance with these driving instruction values, the massage heads 28 will move while deviating from the direction perpendicular to the muscle fiber direction by the angle θ.

Since the massage apparatus of the present embodiment is provided with the above-described force sensor SC and the operation unit P, the memory M is not required to store data about muscle fiber directions of a back. That is, according to the massage apparatus of the present embodiment, the force sensor SC measures a force applied to the massage heads 28 and, instead of the memory M, the operation unit P determines the direction of muscle fibers of the back based on the force components detected by the force sensor SC.

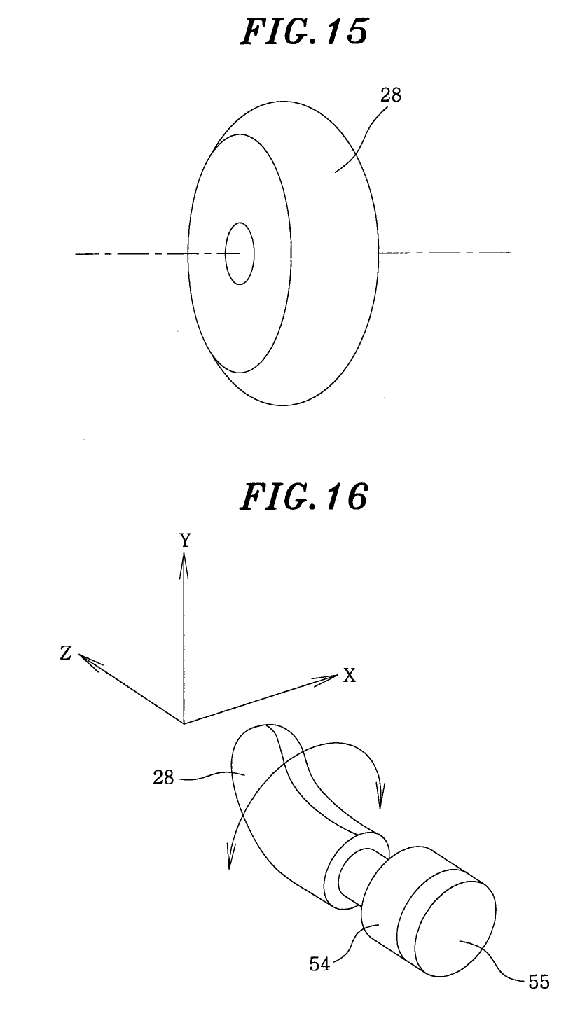

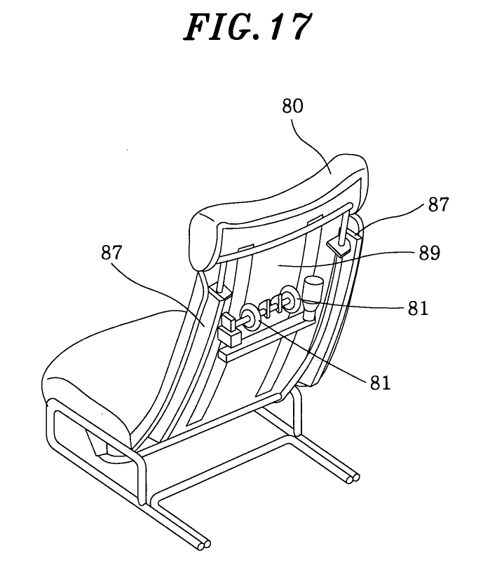

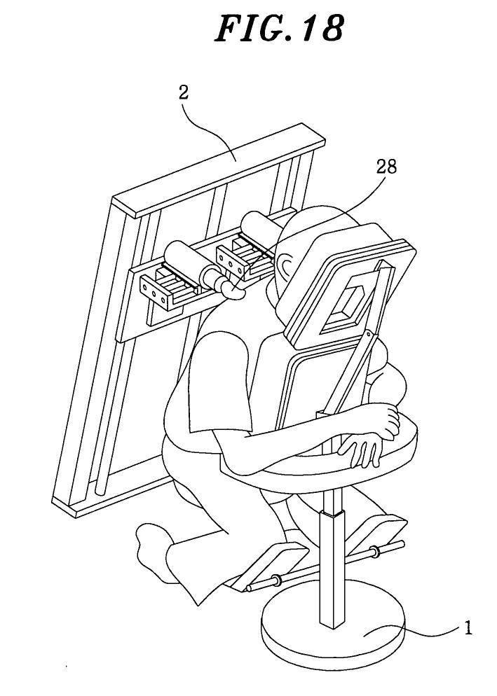

As the massage head 28, a roller shaped massage head may be used as shown in FIG. 15, which is a typical shape for massage heads. However, a massage head having a shape like a human thumb shown in FIG. 16 is preferable so as to enable a more effective massage. The massage head 28 shown in FIG. 16 has an oval contact surface to be in contact with the back. Such shaped massage head 28 is preferable because the massage mechanism 2 can give a massage closer to that by a human massager. In the case of using the thumb-shaped massage head 28, the massage head 28 preferably includes a rotation unit 54 rotating the massage head 28 around Z-axis thereof which is a driving direction by an actuator 27 and an angle detection unit 55 detecting a rotation angle of the massage head 28.

The rotation unit 54 rotates the massage head 28 so that the minor-axis direction of it's oval contact surface agrees with the muscle fiber direction, and the major-axis direction thereof agrees with the direction perpendicular to the muscle fiber direction. In this condition, when the massage head 28 is reciprocated in the direction perpendicular to the muscle fiber direction, the massage will be substantially true reproduction of that performed by a human massager, thus further enhancing the massage effects. Moreover, this massage apparatus enables the massage heads 28 in direct contact with the back of the massage recipient, and therefore can control the applied pressure more accurately as compared with a chair type, for example, in which a massage head is in contact with the back through a back rest cover. Consequently, the massage effects are further enhanced.

This massage apparatus can massage a top surface of the shoulders as well. In this case, the direction of pressing the body surface by the massage heads 28 is aligned with the vertical direction Y of the massage heads 28 moving, whereas the direction perpendicular to the muscle fiber direction is aligned with the projecting direction Z of the massage heads 28. That is, the moving direction of the massage heads 28 along the body surface is in agreement with the pressing direction of the massage heads 28.

As stated above, according to the massage apparatus of the present embodiment, the muscle fiber direction in a surface being along the body surface can be determined accurately based on a ratio of the two-axial components of the force applied to the massage heads 28. The conventional massage apparatuses may apply a force in only a predetermined direction. However, the massage apparatus of the present embodiment can determine directions of muscle fibers which are different in each part of the back. So, if the massage heads 28 are moved in the direction corresponding to the muscle fiber direction, further effective massage will be obtained.

Note that the massage apparatus of the present embodiment is provided with the contour measurement unit 3 that measures the body contour. When combining the information about the body contour obtained by the contour measurement unit 3 and the information about the muscle fiber direction obtained by the operation unit P, the controller C can obtain more accurate information of the muscle fiber direction in each part to be massaged after specifying the part of the body, which is preferable. Alternatively, in the case where it is necessary to obtain only information of the muscle fiber direction in the part to be in contact with the massage head 28 without specifying the part to be massaged, the operation unit P can obtain more accurate information of the muscle fiber direction without relying on the contour measurement unit 3. When the operation unit P obtains the information of the muscle fiber direction, the controller C can reciprocate the massage heads 28 in the direction perpendicular to the muscle fiber direction of the part, thus enabling effective massage. It is noted that the back of the massage recipient indicates the part that is not in contact with the supporting body 1, and includes not only the back of the massage recipient but also another part such as a top face of the shoulders, a side and a back of the neck, a side and a back of the arm, and the waist.

While the present invention has been described with a preferred embodiment, this description is not intended to limit our invention. Various modifications of the embodiment will be apparent to those skilled in the art. It is therefore contemplated that the appended claims will cover any such modifications or embodiments as fall within the spirit and scope of the invention.

### INDUSTRIAL APPLICABILITY

According to the present invention, a contour measurement unit measures body contour of a massage recipient supported by a supporting body, and then a controller gives a massage in accordance with the measured body contour. Thereby, it is possible to not only specify a massage area precisely but also move a massage head more appropriately. Thus, the massage apparatus of the present invention is expected to further enhance massage effects.

1. A massage apparatus, comprising:
a support capable of supporting a front face of a human body;
a massage mechanism that massages a back of the human body supported by the support, the massage mechanism including a massage head disposed with a space kept from the support and being movable toward the support for coming into contact with the back of the human body; a driving source to control a movement of the massage head; and
a contour measurement unit that measures a contour of the human body supported by the support,
wherein the controller drives the driving source to control the movement of the massage head based on measurement values measured by the contour measurement unit.

2. The massage apparatus as set forth in claim 1,
wherein the contour measurement unit comprises a distance calculation unit that calculates a distance between the massage head and the back of the human body supported by the support,
wherein the controller controls the movement of the massage head based on calculation results calculated by the distance calculation unit.

3. The massage apparatus as set forth in claim 1, further comprising a memory that stores information of muscle fiber directions of the back of the human body,
wherein the controller extracts a plurality of characteristic parts of the human body from the measurement values measured by the contour measurement unit, and
the distance calculation unit calculates lengths between the characteristic parts, and
the controller divides the back of the human body into regions based on the calculated lengths between the characteristic parts and the stored information of muscle fiber directions, each of the regions including muscle fibers having substantially a same direction, and controls a moving direction of the massage head for each of the regions.

4. The massage apparatus as set forth in claim 1, further comprising a force sensor measuring a force applied to the massage head,
wherein the controller feeds back measurement valves measured by the force sensor to the driving source to control a driving output of the driving source.

5. The massage apparatus as set forth in claim 4,
wherein the massage head is configured to move in three axial directions, and the force sensor measures each axial component of the force applied to the massage head.

6. The massage apparatus as set forth in claim 5,
wherein the memory stores a predetermined threshold to be compared with measurement values measured by the force sensor, and the controller compares the each axial component measured by the force sensor with the threshold and suppresses a movement of the massage head in an axis direction where the force exceeds the threshold.

7. The massage apparatus as set forth in claim 1, further comprising
a force sensor measuring a force applied to the massage head; and
an operation unit determining a muscle fiber direction of the back of the human body from a force measured by the force sensor,
wherein the massage head is configured to move in three-axial directions, and
the force sensor measures each axial component of a force applied to the massage head, and
the operation unit determines a muscle fiber direction of the back of the human body from two-axial components of the measured force, each direction of the two-axial components being perpendicular to the other component's direction in which the massage head presses the back of the human body.

8. The massage apparatus as set forth in claim 7,
wherein the controller moves the massage head in a direction perpendicular to the muscle fiber direction determined by the operation unit in a plane parallel to the back of the human body.

9. The massage apparatus as set forth in claim 3, further comprising a rotation unit rotating the massage head,
wherein the massage head has an oval contact surface to be in contact with the back of the human body, and the rotation unit supports the massage head rotatably so that a major-axis direction of the contact surface of the massage head agrees with the direction perpendicular to the muscle fiber direction.

10. The massage apparatus as set forth in claim 1, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

11. The massage apparatus as set forth in claim 8, further comprising a rotation unit rotating the massage head,
wherein the massage head has an oval contact surface to be in contact with the back of the human body, and the rotation unit supports the massage head rotatably so that a major-axis direction of the contact surface of the massage head agrees with the direction perpendicular to the muscle fiber direction.

12. The massage apparatus as set forth in claim 2, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

13. The massage apparatus as set forth in claim 3, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

14. The massage apparatus as set forth in claim 4, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

15. The massage apparatus as set forth in claim 5, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

16. The massage apparatus as set forth in claim 6, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

17. The massage apparatus as set forth in claim 7, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

18. The massage apparatus as set forth in claim 8, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.

19. The massage apparatus as set forth in claim 11, further comprising a housing intercepting outside light,
wherein the contour measurement unit comprises an imaging unit that captures an image of the back of the human body to measure a body contour of the human body supported by the support, and
wherein the support, the massage mechanism, and the contour measurement unit are accommodated within the housing.
